(19) United States
(12) Patent Application Publication
(10) Pub. No.: US 2023/0145860 A1
(43) Pub. Date: May 11, 2023

(54) METHODS TO GENERATE VACCINE COMPOSITIONS THAT PRIME HUMAN LEUKOCYTE ANTIGEN CLASS I RESTRICTED CD8 T-CELL RESPONSES AGAINST VIRAL NON-VIRION-INTEGRAL DERIVED EPITOPES

(71) Applicant: Genovie AB, Södertälje (SE)

(72) Inventor: Reagan Micheal Jarvis, Södertälje (SE)

(21) Appl. No.: 17/907,532

(22) PCT Filed: Apr. 27, 2021
(86) PCT No.: PCT/EP2021/060956

§ 371 Date: Sep. 27, 2022

(30) Foreign Application Priority Data
Apr. 27, 2020 (EP) 20171570.3

(51) Int. Cl. A61K 39/12; A61P 31/14; C07K 14/005

(52) U.S. Cl. CPC A61K 39/12; A61P 31/14; C07K 14/005

(57) ABSTRACT

Method for providing a vaccine composition capable of effectively inducing a systemic immune response and/or a localised immune response upon administration, wherein the composition comprises human leukocyte antigen class I (HLAI)-restricted epitopes selected from viral pathogen non-virion-integral proteins (non-VIP) and thus prime a CD8 T-cell response specifically directed against virally infected cells.

### FIELD OF THE INVENTION

The present invention relates to immunology and vaccines. More specifically the present invention relates to methods for preparing vaccine compositions and to vaccine compositions useful against viral pathogens. In particular, the present invention relates to methods to generate an administrable vaccine composition useful to be a preventive or therapeutic agent for viral infections.

### BACKGROUND OF THE INVENTION

Viral Vaccine Compositions

Currently, commercial vaccine preparations are mainly directed against whole viral open reading frames (ORF) that generally seek to prime neutralizing immunoglobulin (Ig) responses to confer immunity against the target viral pathogen. Vaccination strategies have not generally sought to specifically select HLA-restricted T-cell epitopes for inclusion into the vaccine composition. Often characterization of any HLA-restricted epitopes (HRE) is limited to those found in proteins comprising virions, or ‘virion-integral proteins’ (VIP), as T-cell responses against these particular viral ORF products are dominantly observed in viral infections with clinical presentations and thus presumed to confer productive T-cell-mediated cellular immunity. However, the inclusion in vaccine compositions of VIP-derived HRE is often a consequence of the aim to prime B-cell driven Ig responses, and generally has not taken in to consideration the lifecycle of the target virus replication and spread within the infected host, nor how this lifecycle primes and T-cell centric cellular immunity within the host. Therefore, closer consideration of the viral lifecycle, viral immunoevasion and potential for immunopathologies should be carefully considered when attempting to leverage, or circumvent, T-cell immunity in vaccine compositions.

Adaptive Immune Responses to Viral Infections

Clinical presentations of viral infections are often associated with viral immunoevasion and/or virus-induced immunopathologies. These immunopathologies can often be attributed to overexuberant reaction to ‘virally encoded proteins’ (VEP). Production of Ig against VEP during native viral infections with clinical presentations are generally restricted to VIP available on the virion surface for B-cell sampling and maturation against viral Ig epitopes. This is a similar case for the dominant T-cell responses in viral infections with clinical presentations, with patient T-cell often restricted to HRE derived from VIP. In contrast, viral non-virion-integral proteins (non-VIP) often drive sub-dominant T-cell responses due to a combination of viral lifecycle factors and how the host immune system reacts throughout that viral lifecycle. Host immunity is often subject to active immunoevasion of specific innate and adaptive responses, driven by VEP. Indeed, viral non-VIP are generally responsible for virus-driven dysregulation of host innate immune responses and thus alter the course of downstream adaptive immune responses.

Challenges in Human Leukocyte Antigen (HLA) Restricted Epitope Specification for Vaccine Compositions

Specification of viral HRE for inclusion in vaccine compositions has been challenging due to the high HLA haplotype diversity across the population. HLA allelic variants represent the most diverse gene families in higher organisms, where the genetic diversity of HLA genes confers broad potential for diverse HRE presentation at the population level. That is, genetic diversity in HLA genes drives protein-level diversity that in turn drives differential selection of HRE for loading and presentation at the cell surface. The general lack of systematic and accurate tools to analyse the highly complex processing and presentation of specific HRE in a functional manner has limited the systematic and reliable identification of HRE from VEP. Furthermore, the complex T-cell receptor (TCR) driven T-cell recognition of these HRE drives a diverse array of downstream T-cell-centric adaptive immunity and immunotolerance. A lack of systematic biotechnology tools to functionally analyse HRE-specific T-cell responses has further hampered directed analysis of T-cell responses against VEP as to specify their inclusion in vaccine compositions.

HLA Class I and Class II and Associated T-Cell Responses

The two major classes of HLA, class I (HLAI) class II (HLAII), generally provoke CD8 and CD4 T-cell responses, respectively, and are found in all jawed vertebrates. HLAI molecules are presented by all nucleated cells and generally present intracellular proteins processed predominantly through proteasomal degradation and transport of said degradation products to the endoplasmic reticulum (ER). Within the ER, further proteolytic processing may take place in the process of enzymatically driven loading of HLAI molecules with HLAI-HRE and subsequent trafficking to the cell surface for T-cell sampling. HLAI molecules are also presented on the surface of professional antigen presenting cells (APC). Professional APC such as dendritic cells (DC) or macrophage are central mediators of adaptive immunity. Within professional APC, additional processing and trafficking pathways are represented that enable delivery of protein antigen degradation products, derived from sampling of the extracellular environment, to the ER for HLAI cross-representation. In contrast, HLAII molecules are constitutively presented at the surface of only professional APC. HLAII molecules are largely restricted to the presentation of protein degradation products derived from sampling of the extracellular environment in a process that involves endocytosis of extracellular payloads within endocytic vesicles to fusion with distinct vesicular compartments that contain HLAII molecules, along with a range of processing and chaperone enzymes, which direct HLAII molecule loading with HRE prior to export to the cell surface for T-cell sampling.

HLA Class I and CD8 T-Cell Responses

The primary function of the CD8 T-cell compartment is in their role as cytotoxic lymphocytes (CTL), which mediate the execution of various cell death mechanisms upon target cells detected to present cognate HLAI-HRE epitopes that activate the TCR carried by relevant CD8 CTL clones. The detection of HLAI-HRE by CD8 T-cells thus comprises the surveillance of intracellular proteomes of nucleated cells, and thus represents the central mechanism for adaptive cellular immunity to respond to non-self proteins expressed intracellularly. This marks CD8 T-cell responses detecting HLAI-HRE as the primary defence against dysplastic and malignant cell transformation and viral infection, in addition to bacterial, parasite or other intracellular microbial infections. HLAI-HRE are relatively short peptides, with more stringent physiochemical constraints compared to HLAII-HRE. The relatively stringent nature of HLAI-HRE reflects a requirement for tight control of CD8 CTL adaptive responses, as to avoid tissue damage resulting from spurious CD8 CTL reaction against HLAI-HRE derived from self and non-self antigen components.

HLA Class II and CD4 T-Cell Responses

HLAII-HRE directed CD4 T-cell responses are more complex and diverse than HLAI-HRE CD8 T-cell responses. Generally, CD4 T-cells are considered as ‘helper’ cells that serve to coordinate innate and adaptive immune responses through signalling to leukocyte and non-leukocyte cells alike. With regard to adaptive immunity, HLAII-HRE CD4 T-cell responses are necessary to drive B-cell maturation in production of high-affinity Ig responses and are also involved in committing CD8 T-cells to memory cell differentiation, for example. In detecting the HLAII-HRE sampled from extracellular spaces, CD4 T-cells are central to coordinating adaptive immune responses to infections comprising microorganisms that reside extracellularly, primarily through driving progression of humoral Ig responses through B-cell maturation. Regulatory T-cells (Tregs), responsible for immunotolerance against commensal microorganisms and foodstuffs, for example, are also predominantly identifiable as CD4 T-cells that sample HLAII-HRE. These HLAII-HRE CD4 Treg responses also play a key quality control and inflammation resolution role in innate and adaptive immune responses against pathogenic microorganisms, limiting the potential for virus-related immunopathologies, for example.

Viral Lifecycles and Host CD8 T-Cell Responses

Viral infection of host cells results in delivery of the viral genome and expression of encoded ORFs that can be divided into non-VIP and VIP. Generally, non-VIP are responsible for viral replication and immunoevasion of host defences, while VIP are those proteins assembled into de novo virions within infected cells. Immunoevasion mechanisms of various types may also be ascribed to VIP in certain viruses. It can be generally stated that non-VIP are primarily responsible for processing and replication of viral genomes, coordinating viral genome packaging, controlling de novo virion genesis and mediating immunoevasion.

The priming of productive CD8 CTLs against HLAI-HRE expressed by infected cells is central to adaptive immunity to viral infection. Elimination of virally infected cells by CD8 CTL action is considered a key aspect to both viral clearance and establishment of acquired immunity. Infected host cells trigger a range of innate immune responses to attract sampling of HLAI-HRE by CD8 T-cells, whether naïve CD8 T-cells to initiate CD8 CTL adaptive immunity, or memory CD8 T-cells to recall immunity against prior native infection or vaccination. The primary intracellular innate immune response that drives CD8 T-cell recruitment and sampling are the Interferon Type I (IFN-I). pathways

Viral non-VIP are generally broadly expressed within the cytosolic compartment and thus available to the HLAI-HRE processing and presentation mechanisms, while VIP are often insulated from these mechanisms. Insulation of VIPs from HLAI-HRE processing and presentation is particularly evident in enveloped viruses due to sequestration of structural VIPs to membranes and vesicles in the process of de novo virion genesis. Therefore, CD8 CTL responses against viral non-VIP can be considered as a primary driver of viral clearance in many forms of viral infections, via the elimination of productively infected cells.

Professional APCs are strong inducers of HLAI-HRE CD8 CTL responses. Indeed, particularly DC have mechanisms that promote cellular infection so as to drive beneficial CD8 CTL responses. However, in the absence of productive professional APC infection, the dominant priming of CD8 CTL responses can be biased to VIPs through the endocytosis of whole virions, and trafficking of VIP to the ER for HLAI-HRE cross-presentation by the DC. This likely represents the key driver for immunodominant nature of CD8 T-cell responses toward VIP-derived HLAI-HRE in many viral infections with clinical presentations.

Beneficial CD8 T-Cell Host Responses to Viral Infection and Immunoevasion

The most productive CD8 T-cell response to viral infection are those that prime CD8 CTL responses that can ultimately drive elimination of virally infected cells. This is the primary reason that viral infection, and recognition of viral factors and genomes rapidly triggers innate immune responses, with a prominent role for INF-I inducing innate immunity pathways. IFN-I pathways have a key role in promoting the recruitment and activation of CD8 T-cells, and thus promote priming of beneficial CD8 CTL responses against HLAI-HRE presented by productively infected cells.

A prominent mechanism of viral immunoevasion is the dysregulation and/or suppression of innate INF-I responses, with many viral genomes encoding a number of non-VIP that disrupt IFN-I signalling. This promotes viral immunoevasion, particularly with regard to detection of HLAI-HRE in infected cells by CD8 T-cells. Such immunoevasion mechanisms promote escape of virally infected cells from CD8 CTL-driven elimination while viral replication can proceed. This has clear implications for potential non-productive and even detrimental HLAI-HRE CD8 T-cell responses during viral infection.

Non-Productive CD8 T-Cell Responses to Viral Infection

The escape of virally infected cells from CD8 T-cell responses has clear implication for driving non-productive or even detrimental CD8 T-cell responses during viral infection. Importantly, escape of virally infected cells from the CD8 T-cell responses will selectively limit CD8 CTL responses against viral non-VIP-derived HLAI-HRE that would be most beneficial for viral clearance by elimination of infected cells. Critically, this immunoevasion allows infected cells to generate and release new virions and amplify the infection. This invariably results in the availability of increasing numbers of virions in the extracellular spaces for sampling by professional APC, including DC and macrophage, through pinocytosis, endocytosis or phagocytosis. However, now the available HLAI-HRE epitopes must be derived from VIP, rather than the more productive non-VIP-derived HLAI-HRE, unless the professional APC are themselves subject to productive viral infection.

The result of immunoevasion of CD8 CTL responses against non-VIP-derived HLAI-HRE presented by productively infected cells, and the emergence of immunodominant responses against VIP-derived HLAI-HRE primed by professional APC has clear implications for further downstream viral immunoevasion. Firstly, the dominance of professional APC priming of CD8 T-cell responses diverts CD8 CTL reactions away from productive non-VIP-derived HLAI-HRE, and towards VIP-derived HLAI-HRE. Secondly, the immunodominance of VIP-derived HLAI-HRE will prime CD8 CTL responses that promote the elimination of professional APC by CD8 CTL action. Indeed, CD8 CTL mediated emimintion of professional APCs has been considered a factor in resolution of inflammatory responses to viral infections. However, during active viral infection this can be considered a dysregulated immune response that drives depletion of professional APC at infection site, and in draining lymph nodes (DLN), resulting in lowered capacity for overall adaptive immune responses that professional APCs coordinate; critically HLAI-HRE/CD4 T-cell responses. This form of cumulative immunoevasion of adaptive immunity, downstream of the primary immunoevasion of CTL action against productively infected cells, is critical in viral infections with restricted spectrum of host cell infectivity, particularly when productive infection of professional APCs is absent or inefficient.

CD4 T-Cell and Ig Immunoevasion Derived from Dysregulated CD8 CTL Responses

The accumulation of CD8 CTL responses against VIP-derived HLAI-HRE described above, which can drive depletion of professional APCs during infection, can result in a range of downstream effects deleterious to a productive adaptive immune response to viral infection.

Importantly, professional APC are central to HLAII-HRE specific CD4 T-cell responses, which are themselves central to establishment of humoral Ig immunity. Immunodominant VIP-derived HLAI-HRE CD8 CTL responses that eliminate professional APC at the infection site, at the expense of reaction against productive non-VIP-derived HLAI-HRE, also result in reduction of the professional APC platform availability in DLN that are capable of coordinating CD4 T-cell responses. This inevitably results in a reduced capacity to effectively coordinate timely B-cell maturation and generation of high-affinity neutralising Ig that would otherwise assist in viral clearance and ongoing immunity. This may also result in a reduced capacity of CD4 T-cell responses to effectively mediate commitment of CD8 CTL responses to non-VIP-derived HLAI-HRE to memory differentiation and efficient establishment of CD8-centric ongoing immunity.

Ig-Driven Immunopathologies and Vaccine-Associated Disease

Most current vaccine strategies target compositions that provide largely intact VIP, often within whole inactivated or attenuated viruses, with the central aim of priming neutralising antibody production. However, without the support of coordinated HRE T-cells responses, these strategies can often result in poor neutralising antibody titres and incomplete immunity against the target viral pathogen. Furthermore, several vaccine strategies have resulted in exacerbation of immunopathologies in animal and human subjects, which in part may be attributed to dysregulated Ig responses downstream of mis-directed HRE-specific T-cell response towards VIP. In some cases, developmental vaccination strategies and native infection alike have seen immunopathologies related to the production of neutralising antibodies themselves, particularly in infections of the respiratory mucosa where high titre antibody secretion can result in excessive levels of mucous production and obstructive respiratory distress. These vaccine-associated disease phenomena are poorly understood and are challenging to address due to the high complexity of the HRE repertoire during viral infections, and the high inter-individual HLA haplotype diversity, and an incomplete understanding of overall adaptive immune responses to viral infections.

Prospect for Viral Vaccine Compositions Priming Selected CD8 T-Cell Responses

There is abundant evidence to support the key role of CD8 CTL responses in both the prevention of viral infection establishment and clearance of viral infections. Indeed, animal models clearly demonstrate significant protection from bolus viral challenge by antigen-specific CD8 T-cells. The observations outlined above point to vaccine strategies wherein selected non-VIP-derived HLAI-HRE are provided in a vaccine composition in order to selectively prime beneficial CD8 CTL responses. In doing so, the vaccine composition aims to provide front-line defence against invading viruses, while further avoiding potentially non-productive or harmful priming of CD4 and Ig responses that could result in exacerbation of immunopathologies upon subsequent native viral infection.

To date, it has been challenging to analyse HRE-specific T-cell responses with sufficient depth and precision to specify precise HREs for effective vaccine compositions that aim to prime advantageous T-cell responses. Emerging biotechnology methods now permit deep, rapid and accurate functional analysis of HRE in particular, in addition to T-cell responses against these antigens. (See e.g. WO2018083316, WO2018083317, WO2018083339 and WO2018083318).

### SUMMARY OF THE INVENTION

The present invention provides methods to generate an administrable vaccine composition useful to be a preventive or therapeutic agent for viral infections. The present invention particularly provides a vaccine composition capable of effectively inducing a systemic immune response and/or a localised immune response upon administration, wherein the composition comprises human leukocyte antigen class I (HLAI)-restricted epitopes (HLAI-HRE) selected from viral pathogen non-virion-integral proteins (non-VIP) and thus prime a CD8 T-cell response specifically directed against virally infected cells.

The present invention provides a method for the selection of non-VIP-derived HLAI-HRE with which to generate a vaccine composition that selectively primes CD8 CTL responses beneficial for viral immunity and clearance, while simultaneously avoiding non-productive or deleterious CD8 CTL response against VIP-derived HLAI-HRE.

This method comprises selection of HLAI-HRE for inclusion in the vaccine composition that aims to prime selective CD8 CTL responses directed towards virally infected cells, while avoiding spurious CD8 CTL responses driven by viral immunoevasion mechanisms and downstream immunopathologies.

In particular, the present invention provides methods to generate an administrable vaccine composition as a preventive or therapeutic agent for viral infections. The present invention specifically relates to vaccine compositions capable of effectively inducing a systemic immune response and/or a localised immune response upon administration, wherein the composition comprises one or more non-VIP-derived HLAI-HRE selected from the target viral pathogen, while avoiding HLAI-HRE derived form VIP, thus priming a restrictive and highly defined CD8 T-cell response specifically directed against virally infected cells.

In one aspect the present invention provides a method to generate a vaccine composition for use against a viral pathogen, comprising:
a. Identification of non-virion-integral proteins derived Human Leukocyte Antigen class I restricted epitopes (non-VIP-derived HLAI-HRE) from the viral pathogen against which the vaccine composition is desired.
b. Classification of immunogenicty of the identified non-VIP-derived HLAI-HRE in naïve CD8 T-cell populations isolated from donors without prior target virus infection, and/or in memory CD8 T-cell populations from donors with confirmed active, latent or resolved target virus infection.
c. Selection of non-VIP-derived HLAI-HRE with confirmed immunogenicity in naïve donors, or with observed CD8 T-cell responses in donors with confirmed active, latent or resolved target virus infection.
d. Inclusion of the selected non-VIP-derived HLAI-HRE in the vaccine composition.

Immunogenicity of non-VIP-derived HLAI-HRE in naïve donors or those donors with confirmed prior infection should be against HLAI-HRE that are confirmed to be processed and presented in a cellular model that incorporates productive viral infection as to confirm the availability of said epitopes during the normal viral lifecycle within infected cells. That is, the use of singly-expressed viral ORFs, ORF fragments or use of recombinant peptides is not sufficient to confirm immunogenicity of a given non-VIP-derived HLAI-HRE. This may be confirmed independently of the directed analysis of T-cell responses in naïve or infected donors, for example, via the detection of non-VIP-derived HLAI-HRE in a ex vivo cellular model that has been productively or non-productively infected with a viral pathogen.

Systematic comparative analysis of non-VIP-derived HLAI-HRE immune responses may be done in the following human subject classes:
1. Subjects with asymptomatic SARS-CoV-2 infection
2. Subjects with resolved symptomatic SARS-CoV-2 infection
3. Subjects with severe Covid-19 disease
4. Subjects known to be SARS-CoV-2 infection-naïve

Non-VIP-derived HLAI-HRE identified in each subject class have preference for inclusion into vaccine compositions with the order of priority of 1>2>3>4.

In a second aspect the present invention provides a method to vaccinate a human or veterinary subject to provide immunity against a virus, comprising the administration of a vaccine composition prepared by the method as defined above.

These vaccine formulations may have utility in the treatment and prophylaxis of known pathogens, and in the rapid formulation of response to novel pathogens. Furthermore, knowledge of non-VIP-derived HLAI-HRE enables the development of diagnostic procedures for clinical and epidemiological monitoring.

### BRIEF DESCRIPTION OF THE FIGURES

FIG. 1. SARS-CoV-2 initial infection results evasion of innate immunity mediated by viral-encoded proteins and which mediate evasion of early adaptive CD8 T-cells response.

FIG. 2. Established infection during primary immunoevasion results in delayed recruitment of immune responses.

FIG. 3. Late initiation of adaptive immune response results in bias towards professional APCs sampling accumulating virions and in directed T-cell responses against HLA-restricted antigens derived from virion-integral proteins. This is a correctly directed response for HLAII/CD4 helper and B-cell responses. This may be a counterproductive to HLAI/CD8 CTL response, which should be targeting non-virion proteins expressed highly in productively infected cells, rather than those proteins represented in a virion.

FIG. 4. Misdirected CD8 CTL response against HLAI-restricted epitopes from VIP, cross-presented by professional APC during accumulating viremia and tissue damage, results in depletion of local professional APCs and thus to dysregulated T-cell and B-cell responses, which may underpin cytokine storms in severe Covid-19 presentations and lead to overall weak neutralising antibody responses to SARS-CoV-2 infections.

FIG. 5. a) Selected core HLA-A,B,C alleles, B) proportion of each ethnic group who are predicted to carry at least n number of the core alleles set, C) cumulative probability of observing at least n number of core alleles in a given ethnic group.

### DESCRIPTION OF THE INVENTION

The present invention is described in the following items setting out aspects and embodiments:
1. A method to generate a vaccine composition for use against a viral pathogen, comprising:
   a. Identification of non-virion-integral proteins derived Human Leukocyte Antigen class I restricted epitopes (non-VIP-derived HLAI-HRE) from the viral pathogen against which the vaccine composition is desired.
   b. Classification of immunogenicity of the identified non-VIP-derived HLAI-HRE in naïve CD8 T-cell populations isolated from donors without prior target virus infection, and/or in memory CD8 T-cell populations from donors with confirmed active, latent or resolved target virus infection.
   c. Selection of non-VIP-derived HLAI-HRE with confirmed immunogenicity in naïve donors, or with observed CD8 T-cell responses in donors with confirmed active, latent or resolved target virus infection.
   d. Inclusion of the selected non-VIP-derived HLAI-HRE in the vaccine composition.
2. The method according to item 1, wherein multiple non-VIP-derived HLAI-HRE are selected for inclusion in the vaccine composition as to represent one or more HLAI-HRE in a selection of HLAI alleles that represents those alleles carried by at least 60 percent of individuals within the target population for which the vaccine composition is designed.
3. The method according to item 1, wherein one or more non-VIP-derived HLAI-HRE is selected for inclusion in the vaccine composition as to represent one or more HLAI-HRE in a selection of one or more HLAI alleles that is carried by an individual for which the vaccine composition is designed.
4. The method according to any of items 1-3, wherein the vaccine composition comprises one or more vaccination vectors selected from the following:
   a. A recombinant non-replicating or replicating viral vector
   b. A virus-like particle
   c. A recombinant RNA construct, with or without modified nucleotides
   d. A recombinant DNA construct, with or without modified nucleotides
   e. A recombinant protein, with or without modified amino acids
   f. A synthetic polypeptide, with or without modified amino acids
5. The method according to item 4, wherein said one or more vaccination vectors are selected from a, b or c, and wherein the selected non-VIP-derived HLAI-HRE are incorporated into expression constructs that do not allow expression of functional non-VIP proteins in host cells upon vaccine delivery as to avoid immunoevasion activity of said viral non-VIP.
6. The method according to item 5, wherein the provision of selected non-VIP-derived HLAI-HRE is provided by means of one or more of the following:
   a. Introduction of point mutations and/or sequence insertions and/or sequence deletions, within full-length non-VIP ORFs that inactivate protein function
   b. Construction of synthetic nucleic acid sequences comprising non-VIP ORF fragments that encode selected HLAI-HRE in a concatenated construct
   c. Construction of synthetic nucleic acid sequences comprising non-VIP ORF fragments that encode selected HLAI-HRE within a carrier protein sequence
7. The method according to item 4, wherein said one or more vaccination vectors are selected from d or e, and wherein the recombinant protein or synthetic polypeptides comprise one or more non-VIP-derived HLAI-HRE, and protein or polypeptide molecules comprise concatenated HLAI-HRE or encode said HLAI-HRE within a carrier protein or polypeptide.
8. The method according to any of items 1-7, wherein the viral pathogen is selected from the group consisting of Adenovirus, Alphavirus, Arbovirus, Borna Disease, Bunyavirus, Calicivirus, Condyloma Acuminata, Coronavirus, Coxsackievirus, Cytomegalovirus, Dengue fever virus, Contageous Ecthyma, Epstein-Barr virus, Erythema Infectiosum, Hantavirus, Viral Hemorrhagic Fever, Viral Hepatitis, Herpes Simplex virus, Herpes Zoster virus, HIV, Infectious Mononucleosis, Influenza, Lassa Fever virus, Measles, Mumps, Molluscum Contagiosum, Paramyxovirus, Phlebotomus fever, Polyoma-virus, Poxvirus, Retrovirus, Rift Valley Fever, Rubella, Slow Disease virus, Smallpox, Subacute Sclerosing Panencephalitis, Tumor virus infections, West Nile virus, Yellow Fever virus, Rabies virus and Respiratory Syncitial virus.
9. The method according to item 8, wherein the viral pathogen is a coronavirus, such as SARS-Cov2.
10. The method according to any of items 4-9, wherein the one or more vaccine vectors further encode one or more B-cell/Immunoglobulin epitopes as to prime neutralising Ig responses.
11. The method according to any of items 4-10, wherein the one or more vaccine vectors further encode one or more selected HLAII-HRE epitopes as to prime CD4 T-cell responses to support B-cell maturation and neutralising antibody production, and/or promote commitment of non-VIP-derived HLAI-HRE-specific CD8 T-cell responses to memory differentiation.
12. The method according to item 10, wherein the one or more B-cell/Immunoglobulin epitopes are selected from a VIP protein of the target viral pathogen, and are expressed on the surface of the virion.
13. The method according to any of items 10 and 12, wherein the one or more B-cell/Immunoglobulin epitopes have been modified as to remove HLAI-HRE from the VIP as to avoid priming CD8 T-cell responses against VIP proteins upon vaccine delivery.
14. The method according to item 11, wherein the one or more HLAII-HRE may comprise sequences derived from the target viral pathogen, or may be synthetic or naturally occurring HLAII-HRE epitopes that can promote beneficial CD4 T-cell responses to support B-cell maturation and/or CD8 T-cell memory commitment when included in the vaccine composition.
15. The method according to any of items 4 to 10, 13 and 14, wherein the one or more vaccine vectors further comprise one or more vaccine adjuvants.
16. The method according to any of items 4-15, wherein the vaccine vector is further prepared as a vaccine formulation for administration to a human or veterinary subject, wherein said vaccine formulation further comprises pharmacologically suitable excipients.
17. A vaccine composition for use against a viral pathogen, which is prepared by a method as defined in any of items 1-16.
18. A vaccine formulation comprising a vaccine composition as defined in item 17 and at least one pharmaceutically acceptable excipient.
19. The vaccine formulation according to item 18, further comprising at least one vaccine adjuvant.
20. A method to vaccinate a human or veterinary subject to provide immunity against a virus, comprising the administration of a vaccine composition as defined in item 17, a vaccine formulation as defined in any of items 18-19 or a vaccine composition prepared by the method as defined in any of items 1-16.
21. A method to treat a human or veterinary subject suffering from an acute, chronic or latent viral infection by immunizing said human or veterinary subject with a vaccine composition as defined in item 17, a vaccine formulation as defined in any of items 18-19 or a vaccine composition prepared by the method as defined in any of items 1-16 to provoke an immune reaction.
22. A method to elicit a CD8 T-cell response against a viral infection in a human or veterinary subject, comprising the administration of a vaccine composition as defined in item 17, a vaccine formulation as defined in any of items 18-19 or a vaccine composition prepared by the method as defined in any of items 1-16.
23. The method according to any of items 20-22, wherein the route of administration of the vaccine is selected from intramuscular, intranasal, oral, intraperitoneal, subcutaneous, topical, intradermal, and transdermal delivery.
24. The method according to any of items 20-22, wherein the vaccine is administered intranasally.
25. The method according to any of item 20-22, wherein the vaccine is administered intramuscularly.
26. The method according to any of items 20-22, wherein the vaccine is administered intradermally.
27. The method according to any of items 20-26, wherein the vaccine is administered at two separate occasions, at least 7 days apart to represent a prime-boost vaccination strategy.
28. The method according to any of items 20-27, wherein a prime-boost vaccine is administered by the same route.
29. The method according to any of items 20-27, wherein the prime-boost vaccine is delivered intramuscularly, and the boost vaccine is delivered nasally.
30. The method according to any of items 27-29, wherein the prime and boost vaccine composition, vaccine vector and/or vaccine formulation may be the same or different.

### EXAMPLES

### Example 1: Model of Immunopathogenesis of SARS-CoV-2 Infection Exemplifying Advantages of Non-VIP-Derived HLAI-HRE Vaccine Compositions

In December 2019, several cases of pneumonia with unknown etiology were identified in Wuhan, China. These cases of pneumonia were later associated with a novel virus called Severe Acute Respiratory Syndrome Coronavirus-2 (SARS-CoV-2). On 11 Mar. 2020, the World Health Organization announced that SARS-CoV-2 was causing the disease Coronavirus Disease (COVID-19), now considered a pandemic. This disease is characterized by respiratory tract infections that, depending on the severity, can precipitate acute respiratory stress disorder. Aged subjects with SARS-CoV-2 infection show far poorer prognosis compared to younger subjects, which is suggestive of the fact that the diminished capacity of adaptive immune responses in aged subjects plays a role in severity of SARS-CoV-2 infection and progression to COVID-19 and risk of severe complications and mortality. The present example proposes a consensus model for SARS-CoV-2 immunopathogenesis that has direct implications for both determining current and past infection status in non-aged subjects, and for understanding severity of COVID-19 in aged subjects with implications for the design of vaccines compositions that selectively comprise non-VIP-derived HLAI-HRE.

Viral vaccine development has long been limited by a lack of understanding with regard to antigen-specific T-cell responses to both native viral infection and various vaccine modalities. This limited understanding of T-cell responses is due to a general lack of systematic and high-precision tools to identify HLA-restricted epitopes presented from viral proteins. Moreover, a lack of tools for rapid and definitive testing of human T-cell responses to those various HLA-restricted antigens has been a critical limitation.

Several respiratory viral infections have been associated with acute inflammatory responses in the respiratory mucosa, which have often been linked to dysregulated T-cell responses. Historically, RSV, SARS-CoV and even aggressive strains of influenza have been seen to drive severe inflammatory syndromes of the respiratory mucosa. It is now emerging that subjects with SARS-CoV-2 infection who experience the most severe Covid-19 clinical presentations, are suffering cytokine storms that ultimately drive morbidity. These severe immunopathologies are apparently driven by overexuberant T-cell responses during viremia.

Historical experience with vaccine-associated disease exacerbation in attempts to vaccinate against respiratory viral infections point to significant risks in current initiatives to deploy vaccines against the novel coronavirus. RSV has been recognised to have a high unmet medical need for decades, and early attempts to vaccinate against RSV in the 1960s resulted in exacerbation of acute respiratory distress in vaccine recipients upon subsequent native infection. In the decades of research into this phenomenon that followed, antigen-specific CD8 and CD4 T-cell responses have been implicated in driving both viral clearance and immunopathology alike. Similarly, experimental vaccines for SARS-CoV, the earlier novel coronavirus that was responsible for the 2003 SARS outbreak, led to severe inflammatory responses in developmental vaccines studies, which could be partly attributed to anti-spike immunoglobulins.

In support of an outsized role of T-cell responses in Coronavirus infection, studies on SARS-CoV infection suggested weak B-cell responses, but persistent antigen-specific T-cell responses in recovered subjects—a trend reflected in the SARS-COV-2 pandemic. Moreover, a notable feature of SARS upon SARS-CoV and SARS-CoV-2 infection was lymphopenia, presumed to be driven by massive T-cell recruitment to the respiratory mucosa. Taken together with the apparent correlation of lymphopenia and a poor prognosis in Covid-19 patients, and the emerging details of T-cell immunopathologies in severe Covid-19, there is a clear need to precisely analyse T-cell responses in subjects infected with SARS-CoV-2. This is required to assess safety and efficacy of current vaccine candidates, and to directly inform the development next generation vaccines.

Summary of SARS-CoV-2 Immunopathogenesis Model
Initial immunoevasion of CD8 CTL in early stages of infection results in establishment of infection, which is more likely in aged subjects given the known reduced naïve CD8 T-cell repertoires in aged subjects as a result in decline in thymic output. An effective early CD8-mediated response at this stage may clear the infection even asymptomatically, without subsequent production of detectable high-affinity neutralising antibodies, but with the establishment of memory T-cells. It is apparent that otherwise healthy non-aged subjects often present asymptomatic SARS-CoV-2 infection, o very mild symptoms that resolve with low-level and transient Ig responses.
Initial immunoevasion results in escape of ‘non-virion-integral protein’ (non-VIP) HLAI-restricted epitopes from detection, which are ideally required for clearance of virally infected cells.
Danger associated molecular patterns (DAMP) and pathogen associated molecular patterns (PAMP) that accumulate during establishment of infection and epithelial cell death result in the characteristic recruitment of immune cell infiltrates to infected respiratory mucosa.
Initial immune evasion allows accumulation of virions, that are then sampled by professional APCs, which may not themselves be productively infected.
Processing and presentation of virion-integral proteins (VIP) by professional APCs primes HLAI/CD8 responses and HLAII/CD4 responses.
HLAII/CD4 responses that detect VIP initiate CD4 helper responses to support B-cell maturation against B-cell-sampled virion surface proteins, resulting in a productive adaptive immune response that generates neutralising antibodies. In many cases, but not all, infection may resolve as a result.
HLAI/CD8 responses that detect VIP are not ideally suited to target epithelia or other cells with productive viral infections, while those responses are however capable of targeting professional APCs that are cross-presenting HLAI-restricted antigens from virally infected cells.
Misdirected CD8 CTL response against VIP may result in depletion of professional APC at the infection site and in DLN, and thus to a general breakdown of T-cell response coordination and loss of B-cell maturation support.
Poor B-cell responses may explain inconsistent antibody responses among subjects with SARS-CoV-2 infection and those with Covid-19 clinical presentations. It is therefore unreliable to use serology alone in the clinical management of patients and within epidemiology to set policy, since T-cell responses should also be taken into account.
Vaccination strategies that target abundant VIP such as Spike in SARS-CoV-2 could prime counter-productive CD8 CTL responses and exacerbate disease upon native infection, particularly in vaccine recipients that carry HLA alleles that confer sensitivity to such specific HLA-restricted epitope responses.
A vaccination strategy that aims to precisely prime HLAI-restricted CD8 T-cell responses against non-VIP-derived epitopes so as to establish CD8 T-cell memory responses that can drive viral clearance early during virus encounter and infection, without risk of ADE or of VIP-mediated misdirection of deleterious CD8 CTL response would be beneficial.

Detailed Model of SARS-CoV-2 Immunopathogenesis

The present section outlines a 4-step model of dysregulated immune response that is proposed to be initiated by initial immunoevasion of CD8 CTL responses early in infection via virus-mediated suppression of innate immune signalling in infected cells. Subsequent suppression of innate immune signalling in professional APCs is also likely, even in the absence of productive infection of these cells. Overall, the initial immune escape of infected cells redirects a CD8 CTL response against virion-integral proteins (VIPs). Immune escape results in poor clearance of virally infected cells, in addition to the early initiation of CD8 CTL-driven deletion of professional APCs that is usually associated with resolution of inflammatory response upon significant viral clearance. This model of immunopathogenesis would explain many of the clinical observations associated with severe Covid-19 clinical presentations, in addition to asymptomatic or mild disease in young subjects with confirmed SARS-CoV-2 infections. This model has direct implications for understanding the epidemiology of SARS-CoV-2 pandemic, through the detection of CD8 T-cell effector and memory populations so as to more reliably detect current mild or past infections as compared to unreliable serology testing with notably weak and transient Ig responses in young subjects with SARS-CoV-2 infection. This model supports the sole and selective use of non-VIP-derived HLAI-HRE for vaccine compositions for prophylaxis or treatment of SARS-CoV-2 infection, wherein priming of CD8 CTL responses against non-VIP-derived HLAI-HRE are considered necessary and sufficient for protective immunity towards SARS-CoV-2 and a range of other viral infections.

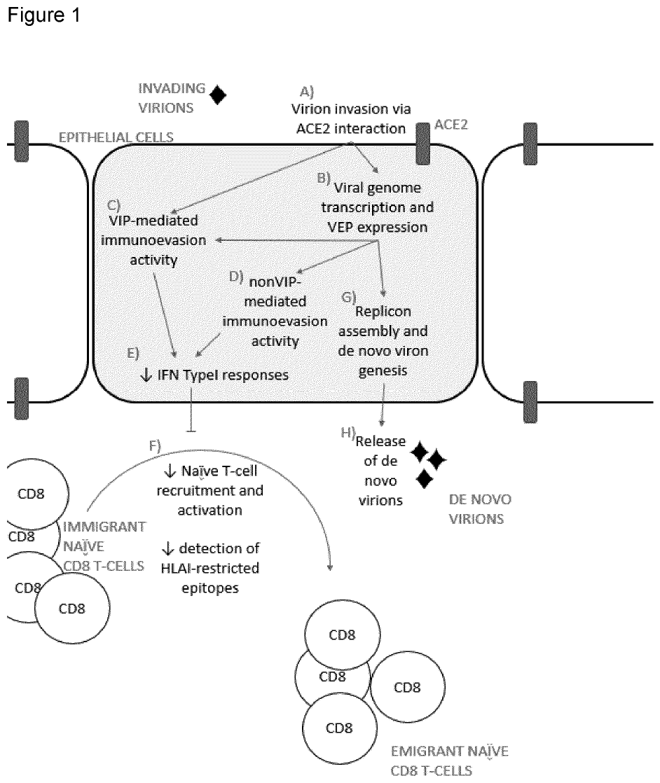

Step 1: Initial SARS-CoV-2 Infection and Immunoevasion of CD8 T-Cells. FIG. 1.

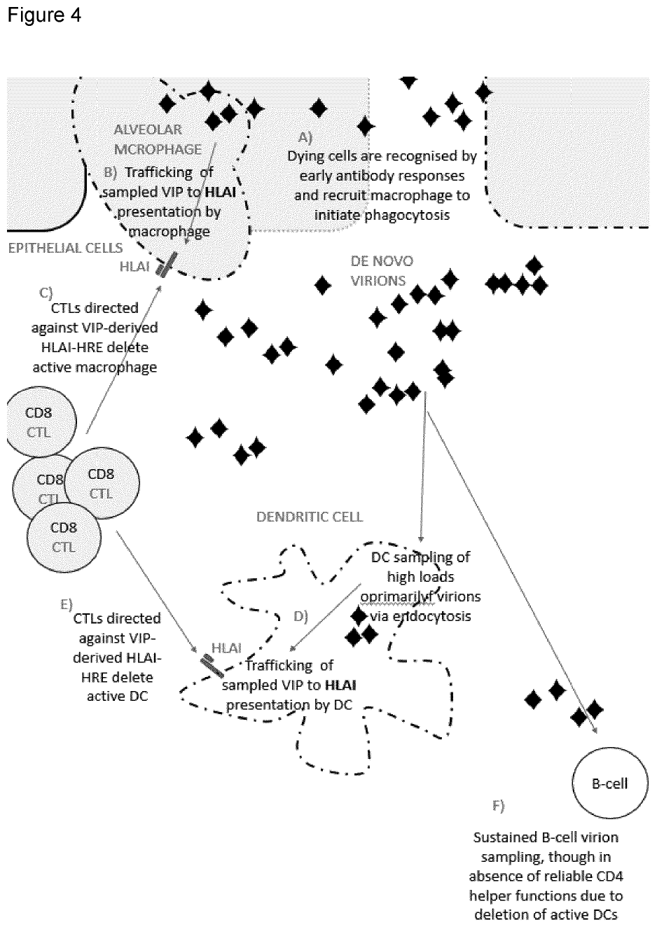

FIG. 4 illustrates SARS-CoV-2 initial infection results evasion of innate immunity mediated by viral-encoded proteins and which mediate evasion of early adaptive CD8 T-cells response.
A) Virion invasion of target epithelial cells occurs through engagement of ACE2 complex and involves furin pre-cleavage in addition to host cell serine protease action, particularly TMPRSS2, for spike complex processing and host cell entry from the endocytic compartment
B) Host cell entry leads to a complex transcription of the viral genome to produce virally encoded proteins (VEP).
C) Virion-integral proteins (VIP) of coronaviruses are known to include factors responsible for immunoevasion activity associated with both structural and non-structural proteins. Delivery of VIP that confer immunoevasion activity is thought to confer an immediate suppression of innate immune responses in the infected cells by delivery of these VIP factors to the host membrane from the virion envelope. Further expression of accessory proteins is driven by transcription of the viral genome to enhance this immunoevasion activity.
D) Non-virion-integral proteins (non-VIP) produced by viral genome transcription may further participate in suppression of host cell innate immunity.
E) A primary means of innate immune suppression driven by VEP is the blockade of IFN Type I (IFNI) responses.
F) Suppression of IFNI responses in infected host cells results in reduced recruitment and sampling of naïve CD8 T-cells, and thus to reduced capacity to detect and respond to viral HLAI-restricted epitopes in the early phases of infection. Thus, the relative availability of naïve and competent memory CD8s is critical to clear early infection at this stage.
G) With immunoevasion of the front line CD8 T-cell responses, infected cells are free to execute viral genome replication and de novo virion genesis.
H) Release of de novo virions from infected cells potentiates further host cell infection.

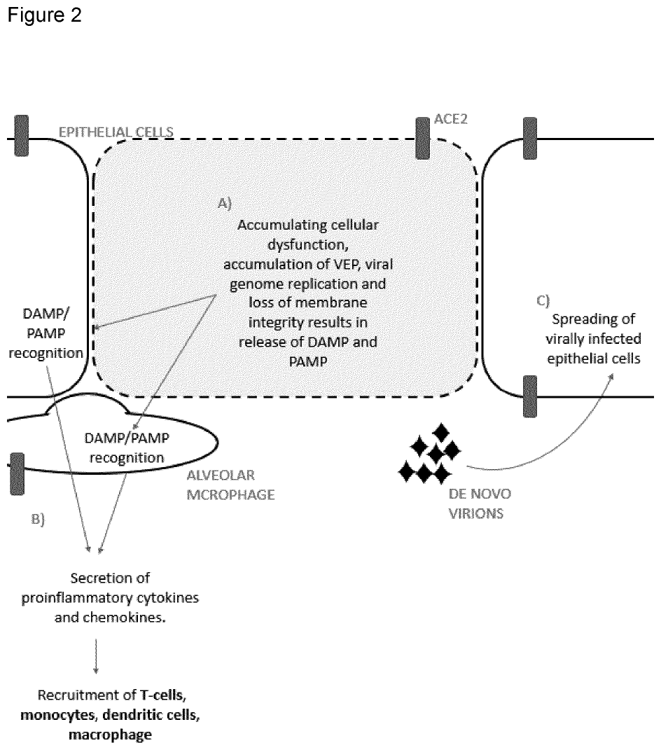

Step 2: Viral Spreading and Immune Activation. FIG. 2.

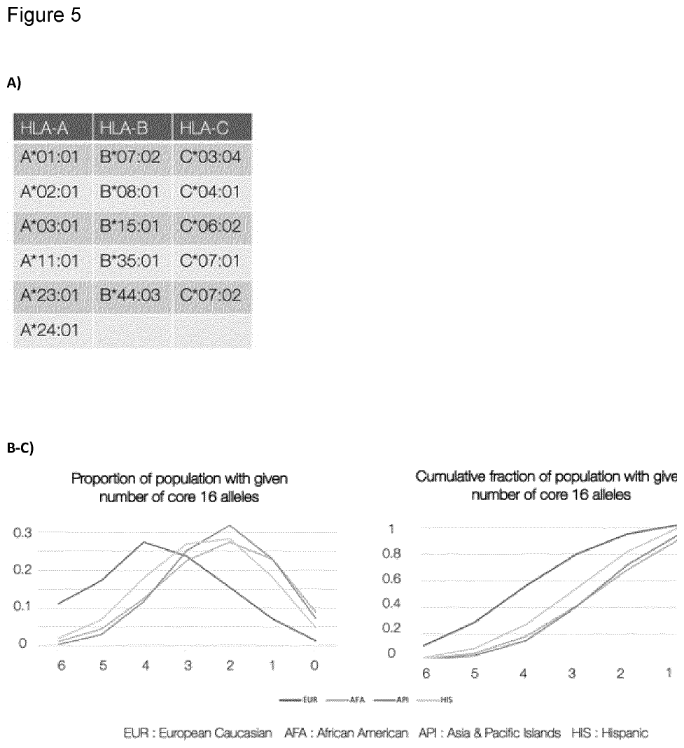

FIG. 5 illustrates established infection during primary immunoevasion results in delayed recruitment of immune responses.
A) Accumulation of cellular dysfunction, VEP expression, viral genome replication and loss of membrane integrity results in the release of danger-associated molecular patterns (DAMP) and pathogen-associated molecular patterns (PAMP).
B) DAMP and PAMP are recognised by innate immunity receptors in both local healthy epithelia and resident alveolar macrophage that induces pro-inflammatory chemokine and cytokine production including; IL-6, MCP1, MIP1a, MIP1b and IP-10.
C) De novo virions released from infected host cells infect adjacent epithelia and initiate viral spreading to other host cells.

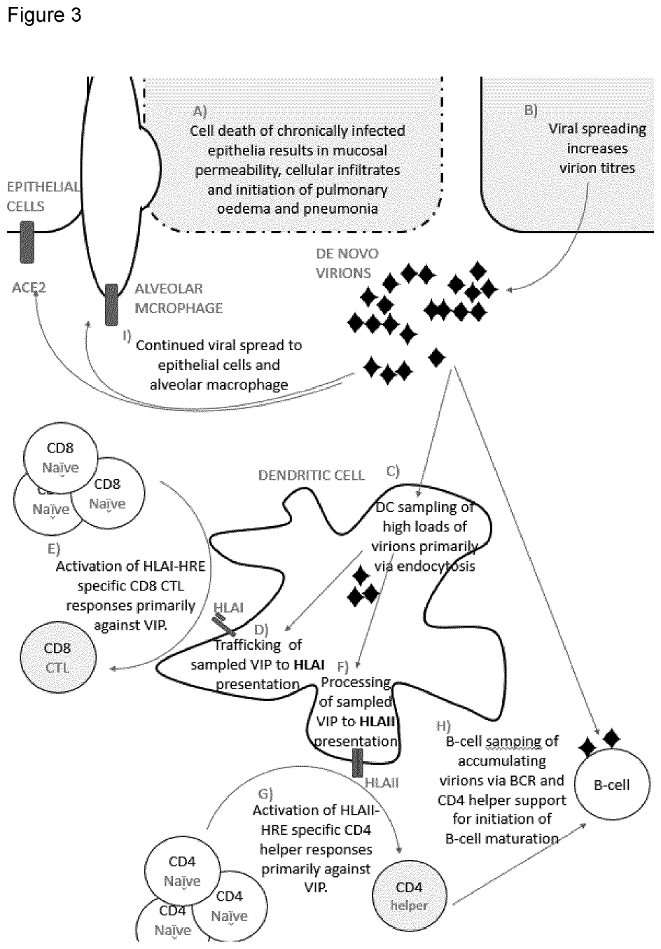

Step 3: Late Initiation of Adaptive Immunity and Misdirected CD8 CTL Response. FIG. 3.

FIG. 6 illustrates late initiation of adaptive immune response results in bias towards professional APCs sampling accumulating virions and in directed T-cell responses against HLA-restricted antigens derived from virion-integral proteins. This is a correctly directed response for HLAII/CD4 helper and B-cell responses. This may be a counterproductive to HLAI/CD8 CTL response, which should be targeting non-virion proteins expressed highly in productively infected cells, rather than those proteins represented in a virion.
A) Cell death of chronically infected epithelial cells results in mucosal permeability, cell infiltration to the pulmonary lumen, oedema and initiation of pneumonia. In particular, alveolar macrophages are important to this process and are responsible for phagocytosis of infected cells and virions.
B) Viral spreading increases virion production within the host and increases extracellular virion titres, allowing further spread of infection and sampling of virions by professional APCs and B-cells to initiate the deeper adaptive immune response that is typical in any form of infection.
C) Sampling of accumulating virions by DC through endocytic mechanisms delivers VIP for processing and presentation of HLA-restricted epitopes. It is unclear whether DC are productively infected, with conflicting reports in the literature. However, it is clear that delivery of virions to DC may impart partial suppression of innate signalling, with a notable lack of activation of DC contacted with virions. This DC activation would usually prime increased HLAI- and HLAII-restricted epitope processing and presentation. In the absence of such activation, the overall detection of HLA-restricted epitopes by CD4 and CD8 T-cells is desensitised—biasing activation of T-cell responses towards the most abundant proteins presented by virions (i.e. VIP).
D) Trafficking of VIP sampled from the extracellular environment to processing and presentation of HLAI-restricted epitopes.
E) Naïve CD8 T-cells sampling DC-presented HLAI-restricted epitopes are activated primarily against VIP, rather than non-VIP, in the absence of productive infection of DCs and in the presence of DC activation suppression. This can be considered an incorrect immune priming, as ideally CD8 CTL-mediated clearance of virally infected cells would target non-VIP abundantly expressed in productively infected cells.
F) Processing of VIP sampled from the extracellular environment leading to presentation of HLAII-restricted epitopes.
G) Naïve CD4 T-cells sampling DC-presented HLAII-restricted epitopes are activated primarily against VIP, rather than non-VIP. This can be considered a correct immune priming, as CD4 helper effector function against VIP are required for B-cell maturation towards neutralising antibody production.
H) B-cell sampling of virions via BCR initiates humoral response with intact CD4 helper effector support, providing first low-affinity antibodies against VIPs, with normal progression of clonal selection and haplotype switching.
I) Further viral spreading to ACE2-expressing cells, notably this apparently includes alveolar macrophage.

Step 4: Misdirected CD8 CTL Response Causes General Immune Dysregulation. FIG. 4.

FIG. 4 illustrates misdirected CD8 CTL response against HLAI-restricted epitopes from VIP, cross-presented by professional APC during accumulating viremia and tissue damage, results in depletion of local professional APCs and thus to dysregulated T-cell and B-cell responses, which may underpin cytokine storms in severe Covid-19 presentations and lead to overall weak neutralising antibody responses to SARS-CoV-2 infections.
A) Apoptotic and necrotic cells are marked by early antibody responses and undergo phagocytosis by macrophage. Further breakdown of the epithelia results in severe tissue damage in the respiratory mucosa and accumulating mucous production along with cell infiltrates, oedema and worsening pneumonia.
B) Materials phagocytosed from macrophage may contribute to VIP-derived HLA-restricted epitopes by presentation via these professional APC. Alveolar macrophage are known to express ACE2, and are thought to be infected. Due to strong innate PAMP receptor expression in macrophage, and generally enhanced innate immune pathways, it is uncertain whether such an infection would be productive under most physiological conditions (i.e. suppression of viral transcription and replication). Regardless, these cells would be rendered susceptible to already primed CTL responses towards VIPs by ongoing phagocytosis of abundant VIP proteins in infected and dying cells, in addition to free virions that are also engulfed.
C) Accumulating CTL responses against VIP-derived HLAI-restricted epitopes may contribute to depletion of macrophages by antigen-specific CTL action during dysregulated immune response.
D) Ongoing DC sampling of virions in viremia sustains VIP-derived HLAI-restricted epitope cross-presentation by these cells.
E) Accumulating CTL responses against VIP-derived HLAI-restricted epitopes may contribute to depletion of macrophages by antigen-specific CTL action during dysregulated immune response, resulting in a lack of professional APCs in MALT and DLN and thus contributing to dysregulated T-cell and B-cell responses that result in accumulating immune dysfunctional and cytokine storms.
F) Depletion of DCs from MALT and DLN results in dysregulated and inefficient CD4 T-cell responses, and a lack of CD4 helper effector function to drive B-cell maturation towards high-quality and diverse neutralising antibody responses.

Implications for Epidemiology of SARS-CoV-2 Infection and Other Viral Infections in Humans.

It is recognised that a significant proportion of subjects with SARS-CoV-2 infection do not generate a quality, or even detectable, antibody response. The mechanisms behind this are unclear, but a dysregulated response B-cell maturation response mediated by a depletion of professional APC by misdirected CD8 CTL activity would account for such unreliable detection of antibody response in even convalescent Covid-19 sufferers.

Moreover, just as phagocytic- and endocytic-sampling by macrophage and DC, respectively, may account for partial immunosuppression of antigen presenting activity, B-cells may be susceptible to virally mediated suppression during viremia. Indeed, the BCR-driven endocytosis by B-cells during viral epitope sampling would entail internalisation of virions that include suppressors of innate immunity expressed in their envelope. This alone could see leakage of immunosuppressive VIP through membrane fusion events between host cell and virion envelope, delivering an innate immune suppression to B-cells that may weaken overall B-cell maturation. Certainly, in the absence of productive infection and destruction of macrophages, DCs or B-cells, the sampling of virions delivers high doses of VIP-derived HLAI-restricted epitopes that may make these professional APC populations susceptible to misdirected CTL action that is deleterious to the overall adaptive immune response by limiting support for antigen-specific CD4 T-cell responses and B-cell maturation.

Numerous studies have linked both productive and non-productive infection of professional APC populations to dysregulated IFNI responses and diminished maturation of professional APCs, which can be linked to direct suppression of innate signalling pathways by VEP. This could contribute to poor overall immunopathogenesis and compound the effect of accumulating adaptive immune dysfunction in subjects with overt COVID-19.

Regardless of the precise mechanisms behind adaptive immune dysregulation, it is clear from emerging reports that serology is unreliable in subjects with asymptomatic or mild Covid-19 presentations. It is furthermore unclear to what extent SARS-CoV-2 infected subjects who are asymptomatic, or present with sub-clinical disease, raise detectable antibody responses. In contrast, it appears likely that most subjects who have cleared subclinical SARS-CoV-2 have done so by raising a sufficient CD8 CTL response directed towards VEP and may never raise an antibody response against VIP.

It is unclear whether subjects with mild infections would raise CD8 CTL responses against other VIP- and/or non-VIP-derived HLA-restricted epitopes. Due to the historical lack in analytical depth of antigen-specific T-cell responses, it is unclear what the role CD8 CTL responses towards particular VIP- and non-VIP-derived HLAI-restricted epitopes contribute to viral clearance in subclinical infections of any virus in human subjects.

Risks of VIP-Encoding Vaccines

It is not the intention herein to account for the generalised risks for adverse events in the deployment of VIP-centric vaccines in the human population. However, with specific regard to the mechanism of immunopathology in severe Covid-19 presentations as posited above, there are specific risks associated with priming sub-neutralising Ig responses in vaccine recipients using VIP-centric compositions, and indeed transient neutralising Ig responses.

Primarily, incomplete or transient Ig-driven protection of vaccine recipients from subsequent native SARS-CoV-2 infections may contribute to vaccine-associated disease exacerbation by two distinct mechanisms. Firstly, a sub-neutralising antibody response may lead to antibody dependent enhancement of Covid-19 via promoting professional APC uptake of partially opsonised (i.e. non-neutralising) virions and promoting misdirected CD8 CTL responses towards VIP-derived HLA-restricted epitopes. Secondly, full-length VIPs (including ‘Spike’ protein) may equally prime CD8 T-cell responses that could lead to accelerated immunopathogenesis and reduced capacity for viral clearance during subsequent native infection.

Even in the presence of productive Ig-driven protective immunity in vaccine recipients with an VIP-encoding vaccine, which would simultaneously prime potentially deleterious CD8 CTL responses against VIP-derived HLAI-HRE, the waning of the Ig titres over time may lead to vaccine-associated disease exacerbation as memory CD8 CTL populations remain in circulation long after resolution of protective Ig titers.

Indeed, it is a fact that despite decades of research there remains a lack of effective vaccines against similar pneumonic viral infections of the lower respiratory tract, such as respiratory syncytial virus (RSV), and only incompletely effective vaccines against influenza, for example. This may be due to a combination of viral vaccine composition and delivery, but in consideration of the observations presented above, this could equally be due to the dual priming of beneficial neutralising antibody responses and of deleterious CD8 CTL responses in inactivated-virus vaccines or recombinant vaccines that deliver VIP with the aim of priming neutralising antibody responses. Historical precedent with vaccine-associated disease exacerbation in RSV vaccination underscores the risks inherent in providing VIP (e.g. through inactivated viral vaccines) as the primary antigenic source during vaccination, which are naturally incapable of providing non-VIP antigenic targets.

In terms of the risks associated with VIP-centric vaccination strategies with regards to the proposed model of immunopathogenesis presented above, there would naturally be a hierarchy of risk with inactivated viral vaccines, full-length VIP and VIP fragment (i.e. gene fragments or protein domains representing epitopes neutralising antibodies) vaccine composition, purely in terms of the sequence space represented in each of these compositions as to provide VIP-derived HLAI-restricted epitopes to prime potentially deleterious CD8 CTL responses that may exacerbate diseases on subsequent native viral infection. Indeed, with the emergent SARS-CoV-2 virus, which has not been widely exposed to the human immune system, there is potential for a high abundance of HLAI-restricted epitopes within any VEP, in the absence of long-standing selective pressure from the human immune system. This implies an enhanced risk for vaccine-associated disease exacerbation in vaccine strategies against SARS-CoV-2, when compared to more established human viral pathogens of a similar nature.

One significant logistical challenge in detecting vaccine-associated disease exacerbation under any circumstances, but particularly in rapid vaccine deployment, is the fact that adverse events are not expected to occur upon initial vaccine challenge, but rather during stochastic subsequent native viral infection. Not only is this a technical challenge to assess in a systematic manner, such is the complexity of T-cell responses to HLA-restricted antigens, but potential adverse events may only become apparent during later phase studies, both due to chronological and probabilistic factors, when a great many subjects have already experienced vaccine challenge. Moreover, such adverse events may occur in the months and years after initial vaccine administration, without subsequent booster vaccination, as VIP-derived HLAI-HRE specific CD8 CTL memory cells persist far beyond significant VIP-directed Ig titres.

Next-Generation Vaccines Compositions Comprised of Non-VIP-Derived HLAI-HRE

With regard to the observations presented above, vaccine compositions comprising selected non-VIP-derived HLAI-HRE is warranted. It is fully acknowledged that the best protection upon vaccination would be provided by a combination of CD8 T-cell memory, neutralising antibody titres and B-cell memory. However, considering the challenges and risks inherent to VIP-centric vaccine strategies for SARS-CoV-2, and indeed other aggressive viral infections of the lower respiratory tract, it is proposed that highly selective inclusion of non-VIP-derived HLAI-HRE is potentially the safest and an adequately efficacious means to deploy vaccines. That is, non-VIP-derived HLAI-HRE are necessary and sufficient to confer protective immunity through selective CD8 CTL responses against SARS-CoV-2 and a range of other human viral pathogens.

Vaccines that do not seek to provoke B-cell responses against VIP, but rather prime non-VIP antigen-specific CD8 T-cell responses, may potentially have an enhanced safety profile. Indeed, due to the complex nature of HLA-restricted antigen presentation in humans, it would be more than reasonable to assume that both vaccine efficacy and vaccine-associated adverse event could be closely predicted by HLAI haplotype of the recipient, and to a lesser extent HLAII haplotype.

In terms of controlling for adverse events, one can certainly quantify T-cell responses towards immunodominant HLA-restricted epitopes, presumably derived from VIP, via established laboratory techniques (i.e. ELISPOT assays with primary specimens) in both patients and vaccine recipients. However, the identification, classification and specification of HLAI-restricted epitopes that provoke sub-dominant T-cell responses in SARS-CoV-2 infection with clinical presentation would be insoluble with such techniques due to poor sensitivity and resolution.

High-resolution and systemised technologies to generate precise analyses of putatively HLAI-restricted epitopes (i.e. non-VIP-derived HLAI-HRE), and their immunogenicity, underpins the potential for next-generation vaccines against the emergent SARS-CoV-2 and viruses with similar infection lifecycles. Considering the limited payload afforded by modern recombinant RNA, DNA and protein vaccine vectors, the compact nature of HLAI-HRE allows maximisation of epitope coverage to match with HLA halplotype of the target population to be vaccinated. Even with the most restrictive recombinant RNA vaccine vectors, at least 60% HLAI allele coverage could be achieved in a worldwide population with a limited collection of non-VIP-derived HLAI-HRE as to confer population-wide protection, particularly in rapid deployment of vaccines against emergent viral pathogens.

In prior studies of similar aggressive viral infections of the respiratory mucosa, several themes have emerged that suggest a potential for dysregulated T-cell immunity, in addition to both T-cell and immunoglobulin-associated immunopathologies.

In an important perspective, acute respiratory distress syndrome (ARDS) has been associated with strong viral immunoglobulin responses in native infection and developmental vaccines. This implies that in persistent infection, exuberant HLAII/CD4 T-cell help drives high-titre antibody production that can be a hallmark of excessive mucous production and respiratory distress. This is potentially related to the infection being poorly controlled by HLAI/CD8 responses in early phase of infection in susceptible subjects, and then high viral titres subsequently driving avid HLAII/CD4 helper responses and B-cell maturation. This fits well with an observed strong correlation of severe Covid-19 presentations with age, and the almost total lack of symptomatic SARS-CoV-2 infection in adolescents and young adults. It is a well-known phenomenon that the naïve T-cell compartment size declines with age, however, there is also an apparent faster relative decline in the naïve CD8 compartment as compared to the naive CD4 compartment. It is possible that older subjects experience immune dysregulation in part due to naturally declining CD8 T-cell response associated with small naïve CD8 T-cell repertoire, and this permits progression of infection and viremia that drives B-cell maturation and immunoglobulin-related immunopathology.

In support of this perspective of the Covid-19 disease progression, subjects who recovered from the related SARS-CoV virus responsible for the 2003 SARS epidemic, were observed to have weak and transitory virus-specific immune responses, lasting just a few months. In contrast, significant CD8 T-cell memory populations were observed to be persistent past a year post-infection in recovered subjects.

Many strategies currently underway for developmental vaccines against SARS-CoV-2 focus on the spike protein as the key antigenic payload. It should be noted, if immunoglobulin-related immunopathologies are responsible for severe Covid-19 inflammatory presentations, this could well be counterproductive. Indeed, this appears to have been the case in similar attempts to vaccinate against other viral infections of the respiratory mucosa.

Moreover, there is an important aspect to the viral lifecycle that would identify the spike protein as a poor reservoir of HLAI-restricted epitopes during native infection. Indeed, the spike protein is membrane-integral, and is produced and inserted into the ER membrane, before trafficking to vesicles in which virion assembly takes place prior to budding. This means that the spike protein is largely insulated from the cytosolic pathways that are the dominant contributors to antigen processing and HLAI-restricted epitope cross-presentation. Not only does this limit potential for productive priming of CD8-centric immunity priming, but the spike protein as such is more likely to drive priming of HLAII/CD4 and immunoglobulin responses that could result in vaccine-induced disease exacerbation upon subsequent native infection

Considering the observations outlined above, it can be hypothesised that immunisation with HLA-restricted epitopes to promote priming of CD8 T-cell immune response represents a safe and effective manner to provide significant protection against SARS-CoV-2 infection. A highly defined payload encoding HLAI-restricted epitopes would serve to establish a CD8 T-cell memory response as front-line protection against native infection, while avoiding potentially detrimental priming of HLAII/CD4 or immunoglobulin responses. Such an approach requires the ability to accurately identify and characterise HLA-restricted epitopes, and the CD8 T-cell responses against these epitopes, in native infection.

One important feature of coronaviruses, and other aggressive viruses that infect the respiratory mucosa, is regulatory proteins encoded in their genomes that disrupt intracellular innate immunity and potentially cause dysregulation of T-cell immunity. Indeed, the SARS-CoV virus responsible for the SARS epidemic in 2003, was found to encode three ORFs capable of disrupting innate immune responses that lead to interferon production, which are similarly contained within the SARS-CoV-2 genome. These cytosolically-available proteins represent ideal targets and natural HLAI-restricted epitope reservoirs during native infection. The ideal design of vaccine payloads would incorporate concatenated ORF fragments from these viral proteins, as to avoid T-cell dysregulation during vaccine delivery, while still promoting strong HLAI-restricted epitope-specific CD8 T-cell responses.

Due to the apparent central role of T-cell immunity in SARS-Cov-2 immunity and immunopathology, the safe and effective deployment of vaccines may have to incorporate targeted immunisation against HLA-restricted epitopes. This is a significant challenge considering the large diversity in haplotypes between ethnic groupings, and geographies. Preparedness for such market or population segmented vaccine designs and deployment represents a significant challenge, while representing a worse-case-scenario of re-emerging SARS-CoV-2 epidemics or pandemics in future, this is a strategic aspect that should not be overlooked.

In an important strategic aspect for consideration is a deeper analysis of HLA-restricted epitopes for selected viral ORFs in the existing core set of HLA alleles, and/or expansion of this analysis to cover distinct strategically important HLAI haplotypes in the global SARS-CoV-2 pandemic.

A critical point in controlling the emergent epidemics moving forward will be the ability to systematically assess immune-escape viral strains that will likely emerge. It should be noted that the novel coronavirus, SARS-CoV-2, has likely emerged from transfer from other animal species to humans, and therefore has not been in widespread contact with, and selective pressure imparted by, the human immune system. It is reasonable to assume that abundant HLAI-restricted epitopes will be presented by the emergent SARS-CoV-2 strain that is driving the pandemic with origins in 2019, and with the widespread infections new strains are likely to emerge that contain mutations that drive escape of specific HLAI-restricted epitope presentation.

### Example 2: Methods for Determination of Immunogenic Non-VIP-Derived HLAI-HRE for Inclusion in Vaccine Compositions

The present example outlines methods with which to select non-VIP-derived HLAI-HRE with which to generate a vaccine composition, using the SARS-CoV-2 viral infection outlined in Example 1.

This section overviews methods to deliver rapid identification and assessment of immunogenicity of HLA-restricted epitopes from the SARS-CoV-2 genome. Analysis of T-cell immunogenicity towards these epitopes is conducted in three different cohorts; patients with severe Covid-19 presentation, subjects with known infection but mild symptoms, and healthy donors naïve to SARS-CoV-2 infection.

The purpose of this overall analysis is to identify HLA-restricted epitopes that most effectively mediate viral clearance, and potentially identify HLA-restricted epitopes that drive dysregulated antigen-specific T-cell responses. These analyses enable next-generation vaccine assets in providing critical data regarding antigen-specific T-cell responses in infection-naïve subjects, critical Covid-19 patients and patients with resolved infections.

These example methods can be broken into 5 key work packages:

Work Package 1 (WP1)

SARS-CoV-2 HLAI-restricted epitope discovery

Work Package 2a (WP2a)

antigen-specific CD8 T-cell activation and TCR screening

Work Package 2b (WP2b)

HLAI multimer and TCR reagent library production

Work Package 3 (WP3)

Validation of HLAI antigen-specific T-cell assays

Work Package 4 (WP4)

Execution of Covid-19 and infection-naïve subject studies

SARS-CoV-2 HLAI-Restricted Epitope Discovery—WP1

Objectives:
Deliver first complete HLAI-restricted epitope scan of all SARS-CoV-2 ORFs to identify functionally processed and cross-presented HLAI-restricted epitopes within the 16 most prevalent alleles across major markets.
Provide foundational dataset to select non-VIP-derived HLAi-HRE for execution of studies in WP2a, WP2b, WP3 and WP4.

Technologies and Methodology:

An engineered antigen-presenting cell (eAPC) system in its most basic form is a production system to rapidly produce eAPC lines expressing target analytes. These analytes simply represent a target HLA allele, and target antigen open reading frames (ORFs) in which target HLA-restricted epitopes are to be identified. This is achieved through standardized donor vectors for HLA and antigen ORF constructs that are paired with genomic receiver sites within functionally engineered immortal cell lines that represent a ‘programmable’ eAPCs. (WO2018083316)

This eAPC platform delivers high-throughput or high-content production of analyte eAPC to feed mass spectrometry (MS) based methodologies to identify HLA-restricted antigens from integrated analyte ORFs within the background of the intrinsic HLAI-restricted repertoire derived from the eAPC proteins themselves.

This enables systematic analysis of analyte ORFs in single HLA backgrounds (i.e. ‘monoallelic’), through direct observation of functionally processed and presented epitopes from analyte sequences. Optionally, a range of expression constructs that incorporate concatenation of minigene analytes, and optional proteasome-targeting motifs to enhance analyte protein cytosolic processing in the expression system may be used. This mass-spectrometry readout is achieved through HLA pull-down from eAPC cell lysates for sample preparation and liquid chromatography fractionation of samples followed by MS identification of HLA-restricted epitopes.

A collection of the 16 most common alleles forms a core working set with which HLA-restricted antigen discovery is conducted. These are selected to capture optimal HLA coverage across major markets, as depicted in FIG. 5.

Deliverables:
Database of SARS-CoV-2 HLA-restricted epitopes presented by the selected 16 core HLA alleles.
Metadata includes relative intensity scores (z-scores) benchmarked against eAPC-intrinsic HLA-restricted epitope repertoires, and score to HLA allele epitope conformity models.

Antigen-Specific CD8 T-Cell Activation and TCR Screening—WP2a

Objectives:
Establish validated immunoreactive HLA-restricted epitopes identified in WP1 through unbiased screening of SARS-Cov-2 infection-naïve and infected subjects.
Assess sensitivity of CD8 T-cell activation-based and tetramer-based assays for identified HLA-restricted SARS-CoV-2 epitopes
Supply material for TCR validation and analytical standards in WP2b

Technologies and Methodology:

The eAPC platform described under WP1 enables reliable and highly defined HLA-restricted antigen presentation to primary CD8 T-cells isolates from infection-naïve donors, or patients. This is a key component of assaying HLA-restricted epitope responses in cell-based assays, an approach that further underpins TCR discovery capabilities. Indeed, ‘monoallelic’ eAPCs represent a robust and reproducible mode of stimulating and testing HLA-restricted epitope responses in T-cell subpopulations, without a need for complex partitioning and culturing of multiple primary cell types from each specimen. This is particularly important in specifying immunogenic HLA-restricted epitopes during native viral infection.

Effectively this work package deploys a set of technologies and methodologies;
1. eAPC-based HLA-allele restricted stimulation of isolated naïve and memory CD8 T-cell populations from SARS-CoV-2 infection-naïve and infected subjects (WO2018083316).
2. HLA-multimer reagent-based quantification of HLA-restricted epitope-specific T-cell response after eAPC-stimulated outgrowth.
3. Single-cell deposition of tetramer-positive cells by flow cytometry for downstream TCR clonotype sequencing and validation
4. eAPC-based re-stimulation of outgrown primary CD8 T-cell isolates to readout HLA-restricted epitope T-cell responses by T-cell activation markers, primarily INFgamma production

The central purpose to this integrated assessment of HLA-restricted epitope immunogenicity is to provide an unbiased landscape of potential immunogenicity to HLA-restricted epitopes encoded in the SARS-CoV-2 genome. This work package will further serve as a preliminary assessment of tetramer-based and cell activation-based flow cytometric assay sensitivity in infection-naïve and infected subjects.

It should be noted that all four techniques may be deployed on a single clinical specimen, which economises use of primary specimens and greatly improves throughput. This is further aided by the routine multiplexing of HLA-multimer reagents in such analyses.

With regard to the re-stimulation approach, the use of systematic eAPC-based HLA allele-restricted stimulation of primary cells, is followed by a re-stimulation procedure with eAPCs constructed from an entirely distinct cell lineage. This is designed to eliminate the background signal in using non-self APCs as stimulation platforms encountered previously.

Deliverables:
Metadata layer on HLAI-restricted epitope database generated in WP1 that details immunoreactive potential of observed HLA-restricted epitopes where assayed.
Preliminary assessment of sensitivity of cell activation-based assays and HLA-based flow cytometric assays for profiling HLA-restricted epitope T-cell responses in SARS-CoV-2 infection.
Validated TCR sequences against immunogenic HLA-restricted epitopes to directly support WP2b, WP3, WP4

HLAI Multimer and TCR Reagent Library Production—WP2b

Objectives:
Construct internally validated HLA-multimer reagent library with matched TCR-expressing engineered TCR-presenting cells (eTPC) analytical standards.
Operationalise the production of all HLA-multimer reagents in the target library to fulfil reagent needs for WP3 and WP4
Generate TCR-expressing eTPC analytical standards that may be utilised in HLA-multimer reagent-based flow cytometry assays deployed in WP3.

Technologies and Methodology:

HLA-multimer production techniques have been highly standardised and are well-known to those skilled in the art, however, these methods comprise challenging protein biochemistry involving protein refolding and purification. The operationalisation of these production workflows is driven by the provision of reliable functional quality control standards in specific TCR-expressing eTPC cell lines.

This work package deploys a range of TCR molecular genetics and cell biology technologies to construct eTPC standards with material derived in WP2a. These technologies can be summarised in four key workflows:
1. Semi-automated TCR alpha/beta chain sequencing and bioinformatic filtering using proprietary primer libraries and software.
2. Rapid PCR-independent TCR alpha/beta ORF reconstitution using a TCR molecular genetics platform—TCR ORF reconstitution and engineering system (TORES)—which enables rapid and cost-effective reconstitution of full-length TCR ORFs within expression vectors (WO2019016175 and WO2018083318).
3. eTPC-based high-throughput screen by integration of paired TCR alpha/beta ORFs to screen for target binding of HLA-multimer reagents and/or interaction with eAPC presening target HLA and HLAI-HRE (WO2018083317 and WO2018083339 and WO2018083318).
4. High-throughput TCR-expressing eTPC standards manufacturing, with stable chemically fixed analytical standard reagent product (WO2018083317).

These methods underpin an ability to generate large high-quality HLA- and TCR-reagent libraries that can be deployed for assay development and execution at unprecedented depth and precision.

Deliverables:
HLA-multimer reagent library for deployment in subsequent work packages
eTPC-based analytical standards library for HLA-multimer reagent manufacturing quality control, and internal controls for flow cytometry assay operationalisation in WP3.

Operationalisation of HLAI Antigen-Specific T-Cell Assays—WP3

Objectives:
Operationalisation of HLA-multimer reagent-based assays for direct staining of peripheral blood, and potentially liquid biopsies, with flow cytometric readout for target HLA allele and restricted epitope coverage to support studies of antigen-specific T-cell responses in WP4.
Operationalisation of eAPC-driven cell activation-based assays for indirect assay readout for parallel deployment in support of WP4.

Technologies and Methodology:

A technological and methodological aspect to approach of scalable deployment of reagent-based assays is the use of TCR-expressing eTPC-based analytical standards. This not only allows for the reliable production quality control of the reagents as specified in WP2b, but also can serve as internal positive control standards to these flow cytometric assays. eTPCs expressing specific TCRs are chemically fixed and stored as stable reagents alongside HLA-multimer reagent stocks for assay execution

The eAPC-driven HLA-restricted epitope stimulation of primary T-cell isolates in cell activation-based assays directly detect the presence of antigen-specific T-cell clones in CD8 T-cell memory compartment for viral epitopes. This assay is also sensitive enough to detect antigen-specific T-cell clones in the naïve compartment. These assays rely on peptide loading of monoallelic eAPC preparations expressing the desired HLA allele, then contact of CD8 T-cell isolates from subject to stimulate memory and/or naïve T-cell compartments. This enables relatively high throughput of analysis for HLA-restricted epitope-specific T-cell responses in both memory and naïve T-cell compartments in parallel, as compared to use of primary APC sources in standard mix leukocyte reactions.

The logistics around both reagent-based and activation-based assay operationalisation in the current work package involves the accurate HLA typing of all subjects, as to assign correct HLA-multimer reagents and/or peptide-loaded monoallelic eAPCs to analysis of individual subjects.

Deliverables:
Operationalised HLA-multimer reagent-based assays to support WP4
Operationalised cell activation-based assays to support WP4 on selected HLA allele and HLA-restricted epitope coverage

Execution of Patient and Infection-Naïve Subject Studies—WP4

This work package aims to deliver systematic studies on non-VIP-derived HLAI-HRE responses in human subjects to determine immunogenic epitopes that drive viral clearance. These studies are enabled by the construction of tools and assays in WP1, WP2a, WP2b and WP3

Objectives:
Execute comparative studies in primary specimens from SARS-CoV-2 infection-naïve and infected subjects as to select immunogenic non-VIP-derived HLAI-HRE suitable for inclusion in vaccine compositions.

Technologies and Methodologies:

The current work package deploys reagents and assays established in preceding work packages and is focused on comparative analysis of antigen-specific T-cell responses in subjects known to have resolved SARS-CoV-2 infection without severe clinical presentations, to subjects with severe Covid-19 presentations. In determining the dominant antigen-specific CD8 T-cell responses in different SARS-CoV-2 outcomes, the central aim is to identify non-VIP-derived HLAI-HRE with confirmed immunogenicity in human subjects to define vaccine compositions. The main specimen type in these studies is peripheral blood from HLA-typed subjects, due to the relative ease in scaling such analyses.

Deliverables:
Delivery of systematic comparative analysis of non-VIP-derived HLAI-HRE immune responses in the following human subject classes:
5. Subjects with asymptomatic SARS-CoV-2 infection
6. Subjects with resolved symptomatic SARS-CoV-2 infection
7. Subjects with severe Covid-19 disease
8. Subjects known to be SARS-CoV-2 infection-naïve

Non-VIP-derived HLAI-HRE identified in each subject class have preference for inclusion into vaccine compositions with the order of 1>2>3>4.

### Definitions

Vaccine

A preparation generally comprising antigen components of a microorganism, virus, or other self or non-self substances, which when administered to a human or veterinary subject aims to provoke an adaptive immune response in the host as to provide immunity or tolerance of the host towards the target microorganism, virus, allergen, or other non-self or self substances.

Vaccine composition

The antigens and adjuvants delivered by a vaccine vector to provoke desired immune responses when said vaccine is formulated and administered to a human or veterinary subject.

Vaccine Vector

A vaccine vector in the present context is defined as the vector by which a vaccine composition is administered to the human or veterinary subject, and includes; recombinant non-replicating or replicating viral vector; virus-like particles; recombinant RNA constructs; recombinant DNA constructs; recombinant protein and/or protein complexes; synthetic polypeptides.

Vaccine Adjuvant

A molecule or compound that possesses intrinsic immunomodulatory properties and, when co-administered with an antigen, effectively potentiates the host antigen-specific immune responses compared to responses raised when antigen is administered alone. Some viral vectors and virus-like particle vectors are considered to possess intrinsic adjuvant molecules and compounds, while additional co-stimulatory molecules may be encoded in nucleic acid, protein and polypeptide sequences, or conjugated to these antigenic biomolecules, which comprise the vaccine composition. Adjuvant molecules and compounds may further be included in the vaccine formulation.

Vaccine formulation

The preparation into which a vaccine vector is formulated for administration to a human or veterinary subject along with pharmacologically suitable vaccine adjuvants and excipients, wherein excipients may include; anti-adherents, binders, coatings, colours, disintegrants, flavours, glidants, lubricants, preservatives, sorbents, sweeteners and vehicles.

Prime-boost vaccination

A consecutive vaccination strategy where a vaccine is delivered to the human or veterinary subject on multiple occasions as to ‘prime’ desired antigen-specific immune responses, and subsequently boost those immune responses with subsequent vaccine administrations. Prime-boost vaccine strategies may be administered in homologous a manner wherein vaccine compositions, vaccine vector and/or vaccine formulation and/or vaccine route may be the same at both prime and boost administrations. Prime-boost vaccine strategies may also be administered in a heterologous a manner wherein vaccine compositions, vaccine vector and/or vaccine formulation and/or vaccine route may be the different at both prime and boost administrations.

Antigen

Molecules, particularly biomolecules represented, which represent targets for the adaptive immune system; wherein antigens are detected by immunoglobulins and T-cell receptors in the context of B-cell and T-cell systems, respectively.

Epitope

An epitope, also known as antigenic determinant, is the part of an antigen that is recognized by the adaptive immune system, particularly by immunoglobulins (Ig) or T-cell receptors (TCR). That is, the epitope is the specific part of the antigen to which an immunoglobulin or T-cell receptor binds.

Human leukocyte antigen (HLA)

The human leukocyte antigen (HLA) system or complex is a gene complex encoding the major histocompatibility complex (MHC) proteins in jawed vertebrates. For clarity, the HLA nomenclature is used herein to describe both the genes (i.e. HLA) and protein complexes (i.e. MHC).

Human leukocyte antigen class I (HLAI)

HLAs corresponding to MHC class I (A, B, C and E), which comprise the HLA Class 1 group.

Human leukocyte antigen class II (HLAII)

HLAs corresponding to MHC class II (DP, DM, DO, DQ, and DR), which comprise the HLA Class 2 group.

HLA-restricted epitope (HRE)

Any epitope loaded in an HLA molecule and presented at the cell surface for T-cell sampling via the TCR. Generally, for HLAI and II molecules, these epitopes represent peptides. Non-peptide molecules may also be loaded into HLAs for presentation at the cell surface.

HLAI-restricted epitope (HLAI-HRE)

Any epitope that many be loaded specifically in an HLAI molecule for export to the cell surface for T-cell sampling.

HLAII-restricted epitopes (HLII-HRE)

Any epitope that many be loaded specifically in an HLAII molecule for export to the cell surface for T-cell sampling.

Virally-encoded protein (VEP)

All proteins encoded in a viral genome, including post-translational modifications of said proteins.

Virion

An entire virus particle.

Virion-integral protein (VIP)

Any protein encoded in a viral genome that is represented within a virion as it is released from infected host cells after de novo virion genesis.

Non-virion-integral protein (non-VIP)

Any protein encoded in a viral genome that is expressed within infected host cells during the virus lifecycle, and not represented within a virion.

Antigen presenting cell

Any cell that expresses HLAI molecules, and able to load HLAI-restricted epitopes and present these complexes to the cell surface for T-cell sampling. Generally, considered to be all nucleated cells of jawed vertebrates.

Professional antigen presenting cell

Specialised APC of the immune system that constitutively express HLAII molecules and incorporate pathways in which sampling of extracellular components, via pinocytosis, endocytosis and phagocytosis, results in HLAII-restricted epitope presentation. The main types of professional APC are dendritic cells, macrophage and B-cells.

T-cell

T-lymphocyte, or T-cell, represents thymically-differentiated lymphocytes that mediate diverse adaptive immune functions by way of detecting antigen epitopes via the T-cell receptor (TCR).

TCR

The defining immune receptors of T-cells that enables detection of antigen epitopes in an adaptive manner. Alpha/beta and gamma/delta TCR systems exist, wherein the alpha/beta is generally responsible for the detection of HLAI and HLAII restricted epitopes.

Naive T-cell

A naive T cell is a T cell that has differentiated in bone marrow, and successfully undergone the positive and negative processes of central selection in the thymus.

Effector T-cell

A CD4 helper or a cytotoxic CD8 T-cell that has been activated by productive epitope encounter, and thus been committed to affect the selected function(s) of said T-cell.

Memory T-cell

Committed effect T-cells may undergo subsequent commitment to a memory state, which can represent a quiescent cell posture until productive epitope re-encounter, and thus represents the central mode of acquired T-cell immunity, or tolerance, against the cognate antigen-deprived epitopes.

CD8 T-cell

A T-cell that expresses alpha/alpha, alpha/beta or beta/beta CD8 receptor dimers at the cell surface and are generally responsible for sampling of HLAI-HRE presented by APC. The effector function of CD8 T-cells is mostly commonly cytotoxic action but can further include regulatory and helper functions.

CD4 T-cell

A T-cell that expresses CD4 receptor at the cell surface and are generally responsible for sampling of HLAII-HRE presented by APC. The effector function of CD4 T-cells is massively diverse incompletely understood, however are considered as ‘helper’ cells that detect diverse antigen epitopes and coordinate the entire innate and adaptive immune system through complex intracellular signalling networks.

Cytotoxic T-lymphocyte (CTL)

A T-cell with cytotoxic effector function, usually represented by CD8 T-cells.

Helper T-cell

A generalised definition of effector T-cell function for those T-cells without cytotoxic effector function, most often referring to CD4 T-cells.

T-regulatory cell (Treg)

A T-cell with an effector function that specifically imparts, as a central effector function, immunotolerance via a range of immunosuppressive signalling, may be represented by CD4 and CD8 T-cells, though most commonly CD4 T-cells.

B-cell

B-lymphocyte, or B-cell, is a lymphocyte differentiated in bone marrow, and successfully undergone the positive and negative selection prior to emigration from bone marrow. B-cells are defined by surface B-cell receptors (BCR), which represent membrane-bound Immunoglobulins (Ig), with similar genetic and protein structures to TCR. BCR are central to detection of Ig epitopes on initial encounter, which triggers a gated B-cell maturation process that requires ongoing epitope availability and range of helper T-cell inputs to proceed, and which results in the production of various forms of soluble immunoglobulins. B-cells may also act as professional antigen presenting cells, particularly in their ability to interface with helper T-cells to receive signaling inputs.

Immunoglobulin (Ig)

Commonly referred to as antibodies, theses soluble adaptive recognition molecules are produced by matured B-cells, and which can mediate a set of effector functions within the host immune response on binding cognate epitopes.

Neutralizing Immunoglobulin (neutralizing Ig)

An immunoglobin against an epitope that is generally derived from a non-self antigen and serves a generalised effector function of pathogen or non-self molecule neutralisation.

Dendritic Cell (DC)

A professional antigen presenting cell, and central mediator of T-cell and B-cell responses.

Macrophage

A professional antigen presenting cell, and central mediator of T-cell and B-cell responses, usually defined by their phagocytic activity.

Self antigen

An antigen derived from the host organism with regard to adaptive immunity.

Non-self antigen

An antigen derived extrinsically from the host organism with regard to adaptive immunity, often referring to antigens from pathogens and allergens, but may also include antigens from foodstuffs and commensal microorganisms.

Interferon class I (IFN-I)

A family of cytokines found in mammals that bind interferon receptors and help regulate the immune system.

Draining lymph nodes (DLN)

The lymph node draining the tissue of interest, often referring to lymph nodes that drain an infected tissue, a tissue containing dysplasia, malignancies, or directly exposed to allergens, commensal microorganisms and/or foodstuff.

Open reading frame (ORF)

The part of the nucleic acid reading frame that can be transcribed to produce an ORE product or gene transcript.

Immunogenicity

A generalised term to indicate the potential for a given antigen, or B-cell or T-cell epitope, to provoke measurable responses in B-cell and T-cell systems, respectively.

Active infection

A viral infection that has ongoing viral replication and release of de novo virions from infected host cells and infection of further host cells.

Resolved infection

A viral infection that has been cleared by the host immune system.

Latent infection

A viral infection that has resulted in suppression of viral replication by the host immune system, and has resulted in maintenance of viral genomic material within host cells that may in future undergo reactivation and de novo virion genesis to establish an active infection,

Clinical presentation

A viral infection with notable anifestations of clinical relevance.

### Figure Boxes

FIG. 1, Box 1:

Aged Subjects

Range of immunodeficiencies; notably a diminished naïve T-cell repertoire.

Aged subjects have lower Naïve CD8 T-cell surveillance in early phase of infection.

Escape from CD8 CTL activation in early phase of infection.

Results in reduced and/or delayed potential to clear virally infected cells.

FIG. 2, Box 1:

Generalised Immune Activation

Cellular damage releases DAMPS and DAMPS recognized by adjacent cells and macrophage to trigger cytokine and chemokine production.

Immune cell recruitment drives more rigorous sampling of cells by T-cell compartment. Arrival of dendritic cells in higher numbers and response to promote CD8 and CD4 HLA-restricted epitope sampling in MALT and DLN.

FIG. 2, Box 2:

Viral Spread

Immunoevasion of front line CD8 CTL activation against viral HLAI-restricted epitopes allows infection establishment.

FIG. 3, Box 1:

Virion Accumulation

Viral spreading in epithelia allows accumulation of virions for further viral spreading and sampling by professional APC and B-cells.

FIG. 3, Box 2:

Adaptive Response

Widespread immune cell recruitment enables adaptive CD8, CD4 and B-cell response biased towards VIP.

FIG. 4, Box 1:

Viremia and Tissue Damage

Phagocytosed epithelial cells provide VEP for cross-presentation to CD8 T-Cells.

High viral titers in respiratory mucosa and draining lymph nodes are sampled by DC for cross-presentation to CD8 CTLs.

FIG. 4, Box 2:

Dysregulated Adaptive Immune Response

An initial immunoevasion based on suppression of innate immune responses results in escape of productively infected cells from early CD8 T-cell detection and delayed CD8 CTL responses.

Establishment of viral infection, cell death, immune recruitment and viremia drive a delayed adaptive CD8 CTL response polarized towards high-abundance VIP-derived HLAI-HRE.

Resulting depletion of professional APC by CD8 CTL cytotoxic function at site of infection and DLN allows rapidly accumulating tissue damage and reduction of DCs to direct CD4 helper response.

Breakdown in the co-ordination of T-cell adaptive immunity results in severe inflammation and cytokine storms in addition to poor CD4 T-cell help for B-cell maturation, resulting in impaired antibody responses.

1. A method to generate a vaccine composition for use against a viral pathogen, comprising:
a. identifying non-virion-integral proteins derived Human Leukocyte Antigen class I restricted epitopes (non-VIP-derived HLAI-HRE) from the viral pathogen against which the vaccine composition is desired using standardized donor vectors for HLA and antigen ORF constructs that are paired with genomic receiver sites within functionally engineered immortal cell lines that represent programmable eAPCs that are then screened by mass spectrometry (MS) based methodologies to identify HLA-restricted antigens from integrated ORFs within the background of the intrinsic HLAI restricted repertoire derived from the eAPC proteins themselves and enables systematic analysis of the ORFs in single HLA backgrounds (monoalleleic),
b. classifying immunogenicity of the identified non-VIP-derived HLAI-HRE in naïve CD8 T-cell populations isolated from donors without prior target virus infection, and/or in memory CD8 T-cell populations from donors with confirmed active, latent or resolved target virus infection,
c. selecting non-VIP-derived HLAI-HRE with confirmed immunogenicity in naïve donors, or with observed CD8 T-cell responses in donors with confirmed active, latent or resolved target virus infection, and
d. providing the selected non-VIP-derived HLAI-HRE in the vaccine composition, wherein the viral pathogen is SARS-CoV-2.

2-15. (canceled)

16. The method according claim 1, wherein multiple non-VIP-derived HLAI-HRE are selected for inclusion in the vaccine composition as to represent one or more HLAI-HRE in a selection of HLAI alleles that represents those alleles carried by at least 60 percent of individuals within the target population for which the vaccine composition is designed.

17. The method according to claim 1, wherein one or more non-VIP-derived HLAI-HRE is selected for inclusion in the vaccine composition as to represent one or more HLAI-HRE in a selection of one or more HLAI alleles that is carried by an individual for which the vaccine composition is designed.

18. The method according to claim 1, wherein the vaccine composition comprises one or more vaccination vectors selected from the following:
a. a recombinant non-replicating or replicating viral vector,
b. a virus-like particle,
c. a recombinant RNA construct, with or without modified nucleotides,
d. a recombinant DNA construct, with or without modified nucleotides,
e. a recombinant protein, with or without modified amino acids, or
f. a synthetic polypeptide, with or without modified amino acids.

19. The method according to claim 18, wherein said one or more vaccination vectors are selected from a, b or c, and wherein the selected non-VIP-derived HLAI-HRE are incorporated into expression constructs that do not allow expression of functional non-VIP proteins in host cells upon vaccine delivery as to avoid immunoevasion activity of said viral non-VIP.

20. The method according to claim 19, wherein the provision of selected non-VIP-derived HLAI-HRE is provided by one or more of the following:
a. introducing point mutations and/or sequence insertions and/or sequence deletions, within full-length non-VIP ORFs that inactivate protein function,
b. constructing synthetic nucleic acid sequences comprising non-VIP ORF fragments that encode selected HLAI-HRE in a concatenated construct, or
c. constructing synthetic nucleic acid sequences comprising non-VIP ORF fragments that encode selected HLAI-HRE within a carrier protein sequence.

21. The method according to claim 18, wherein said one or more vaccination vectors are selected from d or e, and wherein the recombinant protein or synthetic polypeptides comprise one or more non-VIP-derived HLAI-HRE, and protein or polypeptide molecules comprise concatenated HLAI-HRE or encode said HLAI-HRE within a carrier protein or polypeptide.

22. The method according to claim 18, wherein the one or more vaccination vectors further encode one or more B-cell/Immunoglobulin epitopes so as to prime neutralising Ig responses.

23. The method according to claim 18, wherein the one or more vaccination vectors further encode one or more selected HLAII-HRE epitopes so as to prime CD4 T-cell responses to support B-cell maturation and neutralising antibody production, and/or promote commitment of non-VIP-derived HLAI-HRE-specific CD8 T-cell responses to memory differentiation.

24. The method according to claim 22, wherein the one or more B-cell/Immunoglobulin epitopes have been modified to remove HLAI-HRE from the VIP so as to avoid priming CD8 T-cell responses against VIP proteins upon vaccine delivery.

25. The method according to claim 1, wherein step a further includes an engineered TCR-presenting cell (eTPC).

26. The method according to claim 1, wherein the selected non-VIP derived HLAI-HRE in step. d, is included with priority to HLAI-HRE of non-VIP from naïve donors or with observed CD8 T-cell responses in donors with confirmed active, and/or resolved target virus infection.
